(19) United States
(12) Patent Application Publication
(10) Pub. No.: US 2009/0169872 A1
(43) Pub. Date: Jul. 2, 2009

(54) RADIATION CURABLE COATINGS

(75) Inventors: Vadim V. Krongauz, Bartlett, IL (US); Michael T. K. Ling, Vernon Hills, IL (US); Lecon Woo, Libertyville, IL (US)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., Deerfield, IL (US); BAXTER HEALTHCARE S.A., Zurich (CH)

(21) Appl. No.: 12/341,687

(22) Filed: Dec. 22, 2008

Related U.S. Application Data
(60) Provisional application No. 61/017,109, filed on Dec. 27, 2007.

(51) Int. Cl. B32B 25/08; B32B 33/00; B05D 5/08; B05D 3/06; C08L 67/07; C08L 83/00; C08L 75/04; C08F 2/46; A61M 5/31

(52) U.S. Cl. 428/332; 428/412; 428/423.9; 428/446; 428/495; 427/487; 427/491; 427/489; 427/503; 427/496; 427/515; 427/508; 525/418; 525/419; 524/588; 524/590; 524/599; 522/96; 522/172; 522/179; 522/182; 522/57; 522/51; 522/46; 522/33; 522/43; 522/64; 604/218

(57) ABSTRACT

Radiation curable coatings and methods of utilizing the coatings are provided. In a general embodiment, the present disclosure provides a coating composition containing about 15% to about 80% by weight of one or more radiation curable compounds such as polycarbonate acrylate oligomers, polyurethane acrylate oligomer, polyester acrylate oligomers, silicone-based acrylate oligomers, or a combination thereof, and about 10% to about 80% by weight of one or more acrylate monomers.

### CROSS REFERENCE TO RELATED APPLICATIONS

This application claims the benefit of U.S. Provisional Patent Application Ser. No. 61/017,109 filed on Dec. 27, 2007, the entire disclosure of which is hereby incorporated by reference.

### BACKGROUND

The present disclosure relates to coating technologies. More specifically, the present disclosure relates to radiation curable coatings that reduce the friction of the surface to which the coatings are applied.

Syringe tubes are typically made of rigid materials such as glass, polypropylene, copolyesters, or cyclic olefin copolymers (“COCs”) that provide a rigid structure for storing and injecting drugs or medicines. Plungers that are associated with the syringe tubes generally have a rubber end that pushes the drugs, diluents or medicines through a needle located at the injection end of the syringe. When the rubber end of the plunger contacts the inside surface of the syringe tube, a strong friction arises that makes it difficult to push the plunger through the syringe tube.

Lubricants can be used to reduce the friction between the rubber plunger and the syringe tube. For example, a silicon fluid can be used to coat the inner surface of the syringe tube and/or the rubber end of the plunger. However, the silicon fluid can absorb the drugs or medicines that are to be injected with the syringe, thereby reducing their potency and efficacy. The silicon fluid can also be lost during prolonged contact with the surfaces to which it is applied.

### SUMMARY

The present disclosure provides radiation curable coatings and methods of utilizing the coatings. In a general embodiment, the present disclosure provides a coating composition containing about 15% to about 80% by weight of one or more radiation curable compounds such as polycarbonate acrylate oligomers, polyurethane acrylate oligomers, polyester acrylate oligomers, silicone-based acrylate oligomers, or a combination, and about 10% to about 80% by weight of one or more acrylate monomers. The coating composition can be a solution that is coated onto various substrates.

In an embodiment, the coating composition further contains from about 0.1% to about 20% by weight of at least one vinyl reactive agent. In an alternative embodiment, the coating compositions further contains from about 0.1% to about 10% by weight of at least one thiol compound.

In an embodiment, the coating composition is curable by ultraviolet radiation and contains about 0.5% to about 15% of a photoinitiator. The coating composition can further contain one or more ingredients such as stabilizers, chain transfer agents, plasticizers, light stabilizers, ultra-violet light screening compounds, leveling agents, wetting agents, preservatives, or a combination thereof.

The coating composition can also contain one or more adhesion promoters. The adhesion promoter can be an acid functional compound. The acid functional compound can be a phosphoric acid acrylate or methacrylate, acrylic acid, methacrylic acid, or a combination thereof.

In another embodiment, the present disclosure provides a coated rubber product containing a rubber substrate having a cured coating surrounding at least a portion of the rubber substrate. The rubber substrate can be, for example, a syringe plunger. The cured coating contains: 1) about 15% to about 80% by weight of at least one radiation curable compound selected from the group consisting of polycarbonates, polyurethanes, polyester acrylates, silicone-based acrylate oligomers, and combinations thereof, and 2) about 10% to about 80% by weight of at least one acrylate monomer.

The cured coating can have a thickness between about 2.5 μm and about 500 μm. The cured coating can be radiation cured on the rubber substrate. The cured coating can have a maintained adhesion after about 122° C. steam exposure.

The cured coating can further contain about 0.2% to about 5% by weight of pigments, dyes, or a combination thereof. The cured coating can further contain about 0.1% to about 20% of a pre-polymerized filler such as polyvinyl alcohol, polyvinyl butyrate, or a combination thereof.

In an alternative embodiment, the present disclosure provides a syringe including a syringe tube and a rubber plunger having a cured coating surrounding at least a portion of the rubber plunger. The cured coating contains: 1) about 15% to about 80% by weight of at least one radiation curable compound selected from the group consisting of polycarbonates, polyurethanes, polyester acrylates, silicone-based acrylate oligomers, and combinations thereof; and 2) about 10% to about 80% by weight of at least one acrylate monomer.

In an alternative embodiment, the present disclosure provides a method of making a coated rubber product. The method includes providing a rubber substrate, and coating the rubber substrate with a solution containing: 1) about 15% to about 80% by weight of at least one radiation curable compound such as polycarbonates, polyurethanes, polyester acrylates, silicone-based acrylate oligomers, or a combination thereof; and 2) about 10% to about 80% by weight of at least one acrylate monomer.

The method can further include curing the coating with a radiation treatment. For example, the radiation treatment can be ultra-violet light, electron beam radiation, gamma radiation, plasma, or a combination thereof.

An advantage of the present disclosure is to provide an improved coating composition.

Another advantage of the present disclosure is to provide an improved coating for a rubber substrate having good adhesive properties to the rubber substrate.

Still another advantage of the present disclosure is to provide a coating that reduces the friction between a rubber substrate and a rigid plastic.

Yet another advantage of the present disclosure is to provide an improved method of making a radiation curable coating.

Additional features and advantages are described herein, and will be apparent from the following Detailed Description and the figures.

### BRIEF DESCRIPTION OF THE FIGURES

FIG. 1 is a graph illustrating the sliding force between a rubber plunger and a COC barrel using an uncoated rubber plunger and the reduced sliding force due to a silicon lubricated rubber plunger and a radiation curable coating on the rubber plunger in an embodiment of the present disclosure.

### DETAILED DESCRIPTION

As used herein, when a weight percent or range of weight percents is given for “one or more” materials, the weight percent or range refers to the total weight percent of all listed materials present in the composition.

Radiation curable coatings for substrates and methods of making the same are provided. The curable coatings and coating processes can be used, for example, to replace silicone lubricant used to reduce friction in manufactured syringes. Initial stickiness (static friction) can be reduced between a rubber plunger and a COC syringe tube by applying the curable coating to the rubber plunger. The radiation curable coatings of the present disclosure can also provide a range of mechanical and surface properties alterations for a rubber substrate without affecting the mechanical or barrier performance of the rubber substrate.

In a general embodiment, the present disclosure provides a coating composition containing about 15% to about 80% by weight of one or more radiation curable compounds such as polycarbonate acrylate oligomers, polyurethane acrylate oligomer, polyester acrylate oligomers, silicone-based acrylate oligomers, or a combination, and about 10% to about 80% by weight of one or more acrylate monomers. The coating composition can be a solution that is coated onto various substrates. The acrylate monomer can be isobornylacrylate, isobornylmethacrylate, isooctylacrylate, isooctylmethacrylate, dicyclopentadienyl acrylate, dicyclopentadienyl methacrylate, acrylic acid, methacrylic acid, dimethylacrylamide, or a combination thereof.

In an embodiment, the coating composition further contains from about 0.1% to about 20% by weight of at least one vinyl reactive agent. In an alternative embodiment, the coating compositions further contains from about 0.1% to about 10% by weight of at least one thiol compound.

The coating composition can further contain one or more ingredients such as photoinitiators, stabilizers, chain transfer agents, plasticizers, light stabilizers, ultra-violet light screening compounds, leveling agents, wetting agents, preservatives, or a combination thereof. The photoinitiators can be derivatives of mercaptobenzothiazoles, derivatives of mercaptobenzooxazoles, derivatives of benzophenones, derivatives of acetophenones, derivatives of benzoin alkyl ethers, derivatives of benzil ketals, derivatives of monoacylphosphine oxides, derivatives of bisacylphosphine oxides, or a combination thereof. If a photoinitiator is used, it is present in an amount of about 0.5% to about 15% by weight.

The coating composition can also contain one or more adhesion promoters. For example, the adhesion promoter can be an acid functional compound. The acid function compound can be phosphoric acid acrylates, methacrylate, acrylic acid, methacrylic acid, or a combination thereof.

In another embodiment, the present disclosure provides a coated rubber product containing a rubber substrate having a cured coating surrounding at least a portion of the rubber substrate. The cured coating contains: 1) about 15% to about 80% by weight of at least one radiation curable compound selected from the group consisting of polycarbonates, polyurethanes, polyester acrylates, silicone-based acrylate oligomers, and combinations thereof; and 2) about 10% to about 80% by weight of at least one acrylate monomer.

The rubber substrate can be a product made from a rubber such as, for example, poly(isoprene), poly(chlorobutyl), poly(butadiene), etc. In an embodiment, the rubber substrate is a syringe plunger.

The cured coating can be applied to the rubber substrate so that the thickness of the coating is between about 2.5 μm and about 500 μm. The cured coating can be radiation cured on the rubber substrate using any suitable radiation technology. The cured coating can have a maintained adhesion on the rubber substrate after about 122° C. steam exposure.

The cured coating can also contain about 0.2% to about 5% by weight of pigments, dyes, or a combination thereof. The cured coating can further contain about 0.1% to about 20% of a pre-polymerized filler such as polyvinyl alcohol, polyvinyl butyrate, or a combination thereof.

In an alternative embodiment, the present disclosure provides a method of making a coated rubber product. The method includes providing a rubber substrate, and coating the rubber substrate with a coating solution containing: 1) about 15% to about 80% by weight of at least one radiation curable compound such as polycarbonates, polyurethanes, polyester acrylates, silicone-based acrylate oligomers, or a combination thereof; and 2) about 10% to about 80% by weight of at least one acrylate monomer.

The rubber substrate can be coated with the solution using any suitable coating technique. For example, the coating can be applied by spraying or brushing the coating solution onto the rubber substrate and the subjecting the coated rubber substrate to a radiation treatment. The coating can also be applied by dipping a portion or all of the rubber substrate into the coating solution.

The method can further include curing the coating with a suitable radiation treatment. For example, the radiation treatment can be ultra-violet light, electron beam radiation, gamma radiation, plasma, or a combination thereof.

The rubber substrate can be pre-treated by corona discharge, oxygen ion plasma, or argon ion plasma prior to coating. This may further improve the adhesion of the coating to the rubber substrate.

### EXAMPLES

By way of example and not limitation, the following examples are illustrative of various embodiments of the present disclosure. The percentages are given as % of total weight unless stated otherwise.

### Example 1

A radiation curable resin that provides a protective and surface modifying coating for a rubber substrate was developed and tested. Five coating formulations were studied and are listed in Table 1. The coating formulations were applied as solutions and UV-cured on a poly(chlorobutyl) rubber substrate. After curing, the coatings showed a good adhesion to rubber (i.e. difficult to separate adhesively), elongation at break ranging from 20 to 100%, and elasticity modulus of >10 Pa. During friction studies, the coated rubber to COC surface friction coefficients were reduced to approximately 1.2 or lower at 200 g weight of an applied load (See Table 2).

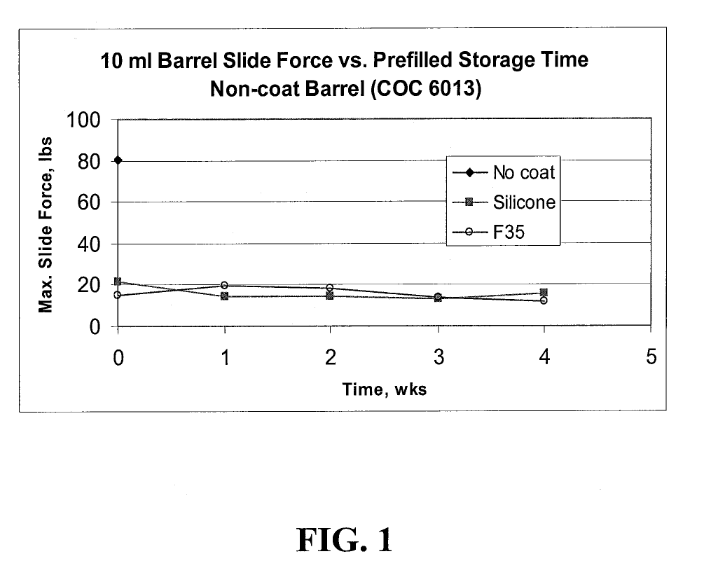

The maximum sliding force was measured between the coated rubber to COC surface over time and is shown in FIG. 1. FIG. 1 illustrates the sliding force between a rubber plunger and a COC barrel using an uncoated rubber plunger compared to the reduced sliding force due to a silicon lubricated rubber plunger and a radiation curable coating (Formulation #1—F35) on the rubber plunger. The results show that the curable coated rubber reduces the sliding force between the rubber plunger and a COC barrel as well as the silicon lubricant. In addition, the curable coated rubber plunger maintains its reduced friction characteristics over a period of four weeks.

TABLE 1
Compositions of Formulations #1-#5

| | | | Example | | | | |
|---|---|---|---|---|---|---|---|
| | Commercial | | #1 | #2 | #3 | #4 | #5 |
| Compound | Name | Function | Weight | Weight | Weight | Weight | Weight |
| α,α-dimethoxy-α- | Irgacure 651 | Initiator | 1.425 | 1.425 | 1.41 | 1.35 | 0.25 |
| phenylacetophenone | (Ciba) | | | | | | |
| Ethoxylated | SR 9035 | Tri-functional | 2.375 | 2.375 | 2.35 | 2.25 | — |
| trimethylolpropane | (Sartomer) | Monomer | | | | | |
| triacrylate | | | | | | | |
| Isobornyl acrylate | SR 506 | Monofunctional | 10.2 | 5.2 | 10.19 | 9.9 | — |
| | (Sartomer) | Monomer | | | | | |
| 1,6 hexanediol | SR 238 | Di-functional | — | 50 | — | — | 8.75 |
| diacrylate (HDODA) | (Sartomer) | Monomer | | | | | |
| Aliphatic diacrylate | CN 132 | Di-functional | — | — | — | — | 11.03 |
| | (Sartomer) | Oligomer | | | | | |
| Siliconized urethane | CN 990 | Di-functional | 9.5 | 9.5 | 9.45 | 9 | — |
| acrylate | (Sartomer) | Oligomer | | | | | |
| Dimethyl acrylamide | Dimethyl | Monofunctional | — | — | — | 2.5 | — |
| | acrylamide | Monomer | | | | | |
| | (Aldrich) | | | | | | |
| Acrylic Acid | Acrylic Acid | | 1.25 | 1.25 | 1.24 | — | — |
| | (Aldrich) | | | | | | |
| N-vinyl pyrrolidone | N-vinyl | Monofunctional | — | — | — | — | 1.27 |
| | pyrrolidone | Monomer | | | | | |
| | (Aldrich) | | | | | | |
| Phosphoric acid | Ebecyl 168 | Adhesion | 0.25 | 0.25 | 0.245 | — | — |
| methacrylate | (UCB | promoter | | | | | |
| | Radcure) | | | | | | |
| Mercaptopropyl | Dynasylan ® | Adhesion | — | — | 0.25 | — | 3.75 |
| trimethoxysilane | MTMO | promoter, | | | | | |
| | (Sivento) | friction reducing | | | | | |
| | | additive | | | | | |

TABLE 2

| | Example | | | | |
|---|---|---|---|---|---|
| | #1 | #2 | #3 | #4 | #5 |
| Contact Angle | 95 | — | 95 | 95 | 66 |
| (degree) | | | | | |
| Elongation at | 39 | 23 | 79 | 91 | — |
| break (%) | | | | | |
| Modulus (psi) | 41254 | 66690 | 27132 | 2546 | — |
| Friction | 0.22 | 1.27 | 1.24 | 2 | — |
| (COC, 200 g) | | | | | |

### CONCLUSION

A series of radiation curable coatings with high elongation, high adhesion to chlorobutyl rubber and low friction relative to a COC material were developed and tested on a rubber substrate. The curable coatings showed low friction (<6 lb) and no detectable water loss at 25° C., 40% relative humidity (RH) in approximately three weeks. It appears that the radiation-curable coatings can be employed in syringes to eliminate the need for liquid silicone-based lubricants currently utilized with chlorobutyl rubber plungers.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A coating composition comprising:
about 15% to about 80% by weight of at least one radiation curable compound selected from the group consisting of polycarbonate acrylate oligomers, polyurethane acrylate oligomer, polyester acrylate oligomers, silicone-based acrylate oligomers, and combinations thereof; and
about 10% to about 80% by weight of at least one acrylate monomer.

2. The coating composition of claim 1 further comprising from about 0.1% to about 20% by weight of at least one vinyl reactive agent.

3. The coating composition of claim 1 further comprising from about 0.1% to about 10% by weight of at least one thiol compound.

4. The coating composition of claim 1, wherein the acrylate monomer is selected from the group consisting of isobornylacrylate, isobornylmethacrylate, isooctylacrylate, isooctylmethacrylate, dicyclopentadienyl acrylate, dicyclopentadienyl methacrylate, acrylic acid, methacrylic acid, dimethylacrylamide and combinations thereof.

5. The coating composition of claim 1 further comprising at least one ingredient selected from the group consisting of stabilizers, chain transfer agents, plasticizers, light stabilizers, ultra-violet light screening compounds, leveling agents, wetting agents, preservatives, and combinations thereof.

6. The coating composition of claim 1 further comprising about 0.5% to about 15% of a photoinitiator.

7. The coating composition of claim 6, wherein the photoinitiators are selected from the group consisting of derivatives of mercaptobenzothiazoles, derivatives of mercaptobenzooxazoles, derivatives of benzophenones, derivatives of acetophenones, derivatives of benzoin alkyl ethers, derivatives of benzil ketals, derivatives of monoacylphosphine oxides, derivatives of bisacylphosphine oxides, and combinations thereof.

8. The coating composition of claim 1 further comprising an adhesion promoter.

9. The coating composition of claim 8, wherein the adhesion promoter is an acid functional compound.

10. The coating composition of claim 9, wherein the acid functional compound is selected from the group consisting of phosphoric acid acrylates, phosphoric acid methacrylates, acrylic acid, methacrylic acid, and combinations thereof.

11. A coated rubber product comprising:
a rubber substrate;
a cured coating surrounding at least a portion of the rubber substrate, the cured coating comprising 1) about 15% to about 80% by weight of at least one radiation curable compound selected from the group consisting of polycarbonates, polyurethanes, polyester acrylates, silicone-based acrylate oligomers, and combinations thereof, and 2) about 10% to about 80% by weight of at least one acrylate monomer.

12. The coated rubber product of claim 11, wherein the cured coating has a thickness between about 2.5 μm and about 500 μm.

13. The coated rubber product of claim 11, wherein the cured coating is radiation cured.

14. The coated rubber product of claim 11, wherein the cured coating further comprises about 0.2% to about 5% by weight of an ingredient selected from the group consisting of pigments, dyes, and combinations thereof.

15. The coated rubber product of claim 11, wherein the cured coating has maintained adhesion after about 122° C. steam exposure.

16. The coated rubber product of claim 11, wherein the cured coating further comprises about 0.1% to about 20% of a pre-polymerized filler selected from the group consisting of polyvinyl alcohol, polyvinyl butyrate, and combinations thereof.

17. The coated rubber product of claim 11, wherein the cured coating comprises from about 0.1% to about 20% by weight of at least one vinyl reactive agent.

18. The coated rubber product of claim 11, wherein the cured coating comprises from about 0.1% to about 10% by weight of at least one thiol compound.

19. The coated rubber product of claim 11, wherein the acrylate monomer is selected from the group consisting of isobornylacrylate, isobornylmethacrylate, isooctylacrylate, isooctylmethacrylate, dicyclopentadienyl acrylate, dicyclopentadienyl methacrylate, acrylic acid, methacrylic acid, dimethylacrylamide, and combinations thereof.

20. The coated rubber product of claim 11, wherein the cured coating comprises at least one ingredient selected from the group consisting of stabilizers, chain transfer agents, plasticizers, light stabilizers, ultra-violet light screening compounds, leveling agents, wetting agents, preservatives, and combinations thereof.

21. The coated rubber product of claim 11 further comprising about 0.5% to about 15% of a photoinitiator.

22. The coated rubber product of claim 11, wherein the cured coating comprises an adhesion promoter.

23. The coated rubber product of claim 11, wherein the rubber substrate is a syringe plunger.

24. A syringe comprising:
a syringe tube; and
a rubber plunger comprising a cured coating surrounding at least a portion of the rubber plunger, the cured coating comprising 1) about 15% to about 80% by weight of at least one radiation curable compound selected from the group consisting of polycarbonates, polyurethanes, polyester acrylates, silicone-based acrylate oligomers, and combinations thereof, and 2) about 10% to about 80% by weight of at least one acrylate monomer.

25. A method of making a coated rubber product, the method comprising:
providing a rubber substrate; and
coating the rubber substrate with a solution comprising 1) about 15% to about 80% by weight of at least one radiation curable compound selected from the group consisting of polycarbonates, polyurethanes, polyester acrylates, silicone-based acrylate oligomers, and combinations thereof, and 2) about 10% to about 80% by weight of at least one acrylate monomer.

26. The method of claim 25 further comprising curing the coating with a radiation treatment.

27. The method of claim 25, wherein the radiation treatment is selected from the group consisting of ultra-violet light, electron beam radiation, gamma radiation, plasma, and combinations thereof.

28. The method of claim 25, wherein the solution further comprises from about 0.1% to about 20% by weight of at least one vinyl reactive agent.

29. The method of claim 25, wherein the solution further comprises from about 0.1% to about 10% by weight of at least one thiol compound.

30. The method of claim 25, wherein the acrylate monomer is selected from the group consisting of isobornylacrylate, isobornylmethacrylate, isooctylacrylate, isooctylmethacrylate, dicyclopentadienyl acrylate, dicyclopentadienyl methacrylate, acrylic acid, methacrylic acid, dimethylacrylamide, and combinations thereof.

31. The method of claim 25, wherein the solution further comprises at least one ingredient selected from the group consisting of photoinitiators, stabilizers, chain transfer agents, plasticizers, light stabilizers, ultra-violet light screening compounds, leveling agents, wetting agents, preservatives, and combinations thereof.
